(19) United States
(12) Patent Application Publication
(10) Pub. No.: US 2010/0094114 A1
(43) Pub. Date: Apr. 15, 2010

(54) USE OF MULTIPLE CALIBRATION SOLUTIONS WITH AN ANALYTE SENSOR WITH USE IN AN AUTOMATED BLOOD ACCESS SYSTEM

(76) Inventors: Mark Ries Robinson, Albuquerque, NM (US); Richard P. Thompson, Dana Point, CA (US)

Correspondence Address: V. Gerald Grafe, esq., P.O. Box 2689, Corrales, NM, 87048, US

(21) Appl. No.: 12/576,303

(22) Filed: Oct. 9, 2009

Related U.S. Application Data
(60) Provisional application No. 61/104,252, filed on Oct. 9, 2008.
(60) Provisional application No. 61/105,600, filed on Oct. 15, 2008.

(51) Int. Cl. A61B 5/145

(52) U.S. Cl. 600/365

(57) ABSTRACT

The invention relates to an automated calibration procedure for analyte sensors such as glucose sensors. The system can provide a calibration point at zero analyte concentration as well as a second calibration point at a known analyte concentration or other pre-determined points. Although not restricted to two point calibration procedure, the system as described enables the system to create one or more calibration points. The use of multiple calibration points can allow the system to correct for both slope and bias drifts. The system also provides the opportunity to provide one or more validation samples. The present invention enables a multitude of options in both calibration and validation to ensure effective operation of the system.

### CROSS REFERENCE TO RELATED APPLICATIONS

This application claims priority to U.S. provisional application 61/105,600, filed Oct. 15, 2008, and to U.S. provisional 61/104,252, filed Oct. 9, 2008, each of which is incorporated herein by reference. This application is related to the following patent applications, each of which is incorporated herein by reference:

U.S. provisional 60/791,719, filed Apr. 12, 2006;
U.S. provisional 60/913,582, filed Apr. 24, 2007;
PCT application PCT/US06/60850, filed Nov. 13, 2006;
U.S. application Ser. No. 11/679,826, filed Feb. 27, 2007;
U.S. application Ser. No. 11/679,837, filed Feb. 28, 2007;
U.S. application Ser. No. 11/679,839, filed Feb. 28, 2007;
U.S. application Ser. No. 11/679,835, filed Feb. 27, 2007;
U.S. application Ser. No. 10/850,646, filed May 21, 2004;
U.S. application Ser. No. 11/842,624, filed Aug. 21, 2007;
U.S. application Ser. No. 12/188,205, filed Aug. 8, 2008;
U.S. provisional 60/991,373, filed Nov. 30, 2007;
U.S. provisional 61/044,004, filed Apr. 10, 2008;
U.S. application Ser. No. 12/108,250, filed Apr. 23, 2008;
U.S. provisional 61/104,252, filed Oct. 9, 2008.

### BACKGROUND

This invention relates to the measurement of blood analytes, and more specifically to the measurement of glucose in blood that has been temporarily removed from the body. Over the past 10 years there has been significant effort devoted to the development of in-vivo glucose sensors that continuously and automatically monitor an individual's glucose level. Such a device enables individuals to more easily monitor their glucose levels. Most of the efforts associated with continuous glucose monitoring have been focused on subcutaneous glucose measurements. In these systems, the measurement device is implanted into the tissue of the individual. The device then reads out a glucose concentration based upon the glucose concentration of the fluid in contact with the measurement device. Most of such systems implant a needle in the subcutaneous space and measure interstitial fluid.

As used herein, a contact glucose sensor is any measurement device that makes physical contact with a fluid containing the glucose to be measured. An example of a contact glucose sensor is an electrochemical sensor. A noncontact glucose sensor is any measurement method that does not require physical contact with the fluid containing the glucose under measurement. Example noncontact glucose sensors include sensors based upon spectroscopy, meaning sensors based on the interaction between light and matter. For the purposes of this application “glucose sensor” includes both contact sensors and noncontact sensors.

Almost all types of glucose sensors are subject to drift over time. Therefore the ability to periodically calibrate these sensors is often desired and necessary. Within the context of automated blood glucose measurements for use in the intensive care unit, a simple and easy to use calibration procedure is desired. Such a calibration procedure should not require nurse intervention and should maintain the overall sterility of the device. Calibration techniques that infuse excessive amounts of glucose into a patient can be undesirable (since maintenance of tight glycemic control is important in many medical settings, including OR and ICU settings).

In the case where the sensor drifts over time, a bias and slope correction can require subsequent validation. The use of bias and slope adjustments to improve calibration or prediction statistics for multivariate models is appropriate provided that the calibration is fully revalidated whenever bias and slope is adjusted. Bias and slope adjustments are another form of calibration transfer and use of bias and slope adjustments can be handled in the same fashion as any other calibration transfer. Prediction errors requiring continued bias and slope corrections indicate drift in reference method or changes in the character of the samples, sample handling, sample presentation, instrument response function, or wavelength stability. If a calibration model fails during the QC monitoring step, the performance of the instrument can be evaluated using the appropriate ASTM instrument performance test [E1944-98 (reapproved 2007), incorporated herein by reference], and any instrument problem that is identified can be corrected. If control samples are used, checks can be performed on the reference method to ensure that reference values are correct. If instrument maintenance is performed, calibration transfer or instrument standardization procedures, or both, can be followed to reestablish the calibration. The preceding information is cited from ASTM International E 1655-05, “Standard Practices for Infrared Multivariate Quantitative Analysis,” Copyright© ASTM International, 100 Barr Harbor Drive, PO Box C700, West Conshohocken, Pa. 19428-2959, United States, 2007, incorporated herein by reference.

In general terms, the ability to calibrate a system and provide subsequent validation is a desired attribute of a blood analyte system. When evaluating an analyte sensor that has multiple analytes, a multitude of calibration samples can be needed to create confidence in the calibration and validation procedure.

### SUMMARY OF THE INVENTION

The invention relates to an automated calibration procedure for analyte sensors such as glucose sensors. The system can provide a calibration point at zero analyte concentration as well as a second calibration point at a known analyte concentration or other pre-determined points. Although not restricted to two point calibration procedure, the system as described enables the system to create one or more calibration points. The use of multiple calibration points can allow the system to correct for both slope and bias drifts. The system also provides the opportunity to provide one or more validation samples. The present invention enables a multitude of options in both calibration and validation to ensure effective operation of the system.

The basis for calibration is the use of maintenance fluids that can be used for calibration. These maintenance fluids can contain known glucose concentrations and can also contain additional additives that improve the overall performance of the system. As examples, specific additives that can be contained in the maintenance fluid include additives that reduce bubble formation, facilitate cleaning of the circuit, reduce protein buildup on the sensing element, or reduce cellular aggregation or platelet adhesion to the circuit. As examples, heparin and citrate can be used as additives that reduce the possibility of cellular aggregation. In the case of heparin, it can be added to either a saline bag or a maintenance fluid bag. Due to the calcium binding effects of citrate, citrate might be added to either the saline or maintenance bag while calcium is added to the other bag. Such a methodology can provide for anticoagulation of the blood while also providing a means for replacing any bound calcium by the direct infusion of calcium during the administration of maintenance fluid to keep the access site open. One of ordinary skill in the art will recognize that a number of additional additives can be placed in the maintenance fluid for the overall improvement or control of system operation.

As used in this application, saline fluids or maintenance fluids are not intended to be restricted to only normal saline but further include any fluid it that is commonly administered to patients in environments such as the intensive care unit. Examples of such fluids include (but are not limited to) normal saline, ½ normal saline, and lactated ringers. In general terms, saline fluid is the fluid used to maintain the patency of the access site. Typically, access sites are in fused in a “keep vein open” or KVO manner at about 3 to 5 ml/hour. The maintenance fluid is typically considered as a secondary fluid designed specifically to facilitate calibration or the overall operation of the device. These general terms are not intended to be restrictive but to provide a better context for the following descriptions. In some applications it is undesirable to infuse a significant amount of one or more of fluids useful in calibrating an analyte sensor. In some applications, for example where a calibration fluid is harmful to the patient, it can be important to infuse zero or nearly zero volume of such a fluid. In other applications, some of such fluid can be infused, but it is important to maintain the amount infused less than an amount that would cause harm to the patient, for example infusion of excessive glucose can be harmful.

An important advantage of the present invention is the ability to perform sensor recalibration in a completely sterile manner. Infection risks within intensive care unit patients are extremely high. The present invention can provide a calibration procedure that does not require “opening” of the system to potential bacteria.

The present invention affords the opportunity for calibrating with known glucose concentrations in saline-based solutions, and for calibrating with blood-based solutions. Some glucose sensing systems can perform differently when exposed to blood, making saline-based calibrations less effective. Many medical products require validation following calibration of the device. The ability to calibrate with saline solutions and subsequently validate performance by additions of glucose to a blood sample is an important advantage of the present invention and potentially provides advantages in the context of CLIA. One of skill in the art will recognize that calibration can be performed on the blood samples with validation on the saline samples and vice versa.

Clinical accuracy needs often dictate higher levels of performance at low glucose levels, often referred to as hypoglycemia, but linearity of response to high glucose levels is also desired. End-users will expect very accurate measurements at hypoglycemic levels and will also expect good linearity over the range of 50 mg/dl to 500 mg/dl. The ability to tailor the calibration procedure based upon the functional sensitivity of the measurement system is a desired aspect of any calibration system. In practice this may require multiple calibration samples at low glucose levels and also calibration samples with high glucose concentrations. The present invention can address these calibration needs by providing the opportunity to use multiple saline-based glucose concentration samples as well as providing the opportunity to create a variety of relative glucose changes in a blood sample. The present invention can provide a variety of calibration procedures.

The present invention provides the opportunity to provide calibration and validation solutions to an array of sensors or to a sensor that measures multiple analytes. The use of multiple solutions containing a variety of concentration levels provides for calibration and validation system in a manner that provides increased confidence. In some cases a solution may be for calibration of one analyte while the same solution serves as a validation solution for another analyte. In some circumstances, a method of additions process can be applied to create accurate calibrations. A variety of implementations can be suitable, including addition of a defined change to an unknown sample.

### BRIEF DESCRIPTION OF THE DRAWINGS

FIG. 1 is an illustration of an example embodiment of a blood access and measurement system suitable for use with the present invention.

FIG. 2 is an illustration of an example embodiment of a blood access and measurement system suitable for use with the present invention.

FIG. 3 is an illustration of an example embodiment where the sensor is located near the patient.

FIG. 3 is an illustration of an example embodiment allowing multilevel calibration.

FIG. 5 is an illustration of an example embodiment which enables mixing of glucose into blood obtained from the patient.

FIG. 6 is an illustration of an example embodiment which enables mixing of glucose into blood obtained from the patient.

FIG. 7 is an illustration of an example embodiment of a blood access and measurement system suitable for use with the present invention.

FIG. 8 is an illustration of an example implementation of a multi-level sensor calibration system.

FIG. 9 is an illustration of an example embodiment which enables mixing of glucose into blood obtained from the patient.

FIG. 10 is an illustration of an example embodiment which enables mixing of glucose into blood obtained from the patient.

FIG. 11 is an illustration of an example embodiment where the sensor is located near patient and where the tube junction between the blood pump and saline pump is located distal the sensor.

FIG. 12 is an illustration of an example of how a relative addition to a sample of unknown glucose concentration can be used to calibrate a system.

FIG. 13 is an illustration of an example of methods of additions.

FIG. 14 is an illustration of an example of methods of additions.

FIG. 15 is an illustration of an example of methods of additions.

FIG. 16 is an illustration of an example of methods of additions.

### EXAMPLE EMBODIMENTS OF THE PRESENT INVENTION

The present invention is described herein in the context of example blood access and measurement systems, for convenience of description. The present invention can also be used in combination with other blood access systems, such as those described in the applications incorporated by reference above.

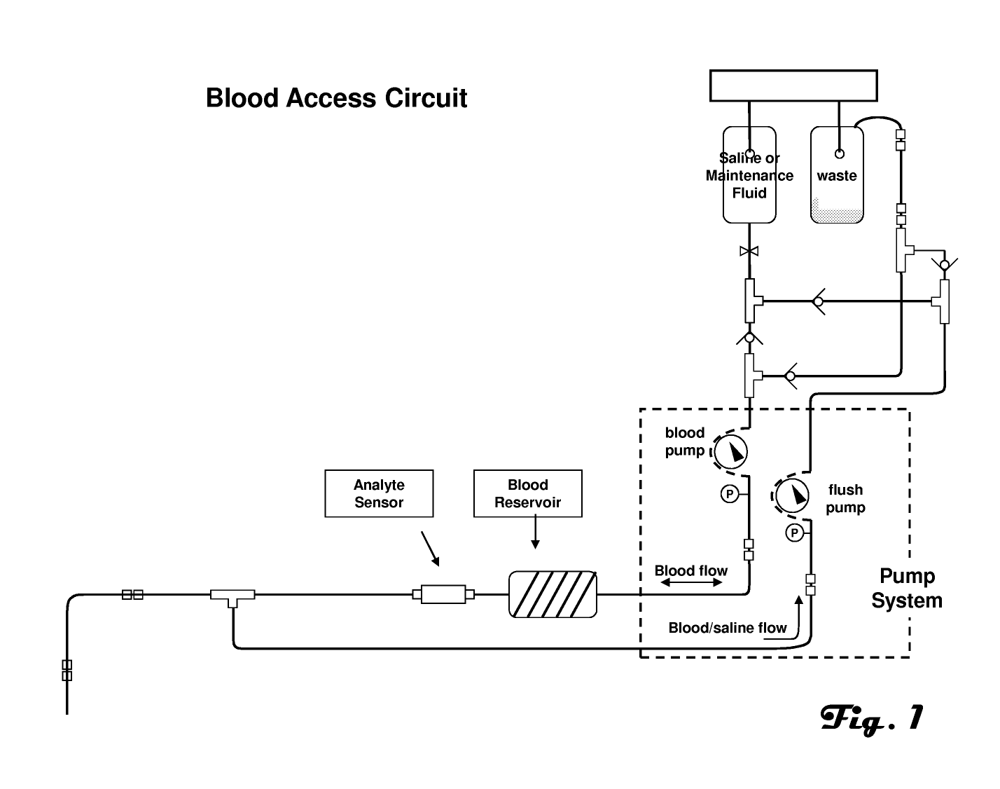

FIG. 1 is an illustration of an example embodiment of a blood access and measurement system suitable for use with the present invention. The example automated blood analyte measurement system contains a sterile fluid solution and a waste bag. The saline or maintenance fluid can contain either zero glucose concentration or a known glucose concentration. Such a system provides the glucose sensor with a known calibration point. In use the sensor can be exposed to this known concentration on a periodic basis.

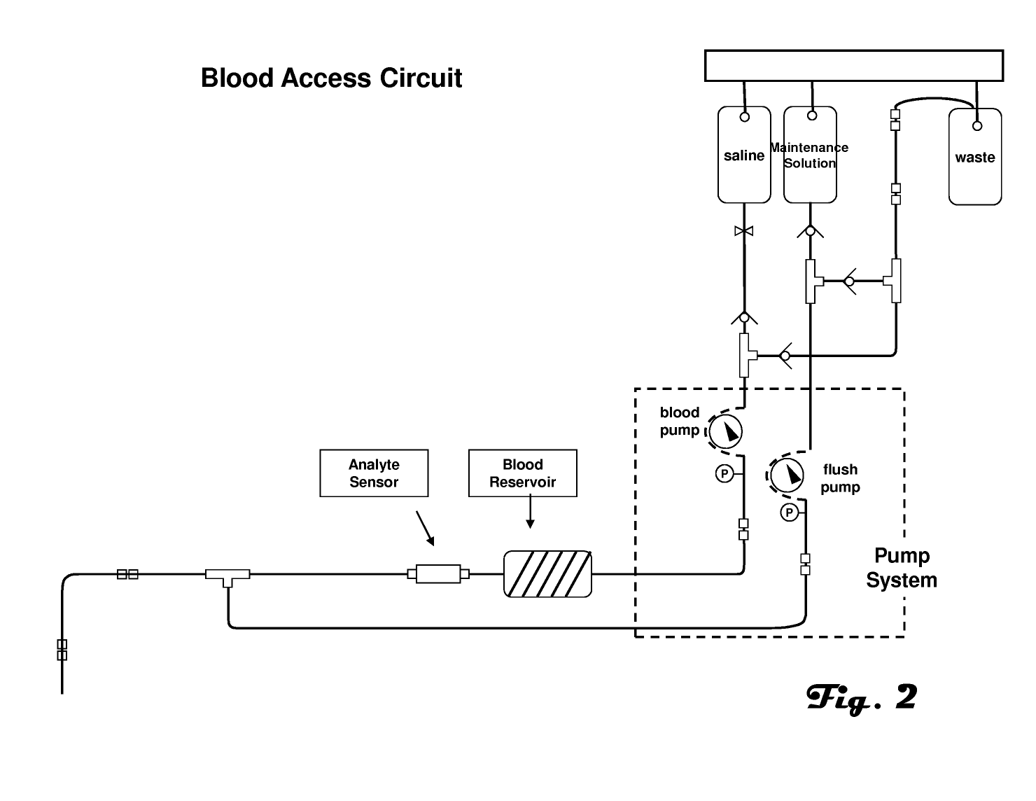

FIG. 2 is an illustration of an example embodiment of a blood access and measurement system suitable for use with the present invention. The example automated blood analyte measurement system contains two fluid bags providing for at least two different calibration points. In use, the analyte sensor can be exposed to a zero or predetermined glucose concentration via fluid from the saline bag. A second glucose concentration can be provided via fluid from the maintenance solution bag. The example system in FIG. 2 provides the opportunity for calibration of the device with a known maintenance fluid while concurrently minimizing the infusion of the maintenance fluid into the patient. In the example system, the maintenance fluid solution can be pumped through the circuit and directly to waste without infusion into the patient. For example, the flush pump can be operated in a manner towards the patient and the blood pump can operate at a similar rate away from the patient. In this manner the analyte sensor is exposed to the maintenance fluid solution but little or no fluid is infused into the patient. Following sensor calibration, fluid from the saline bag can be used to wash the circuit in a similar manner. Such a process can enables the effective calibration of the glucose sensor. Such a system also provides the opportunity to clean or maintain circuit performance with additives where infusion into the subject is not desired.

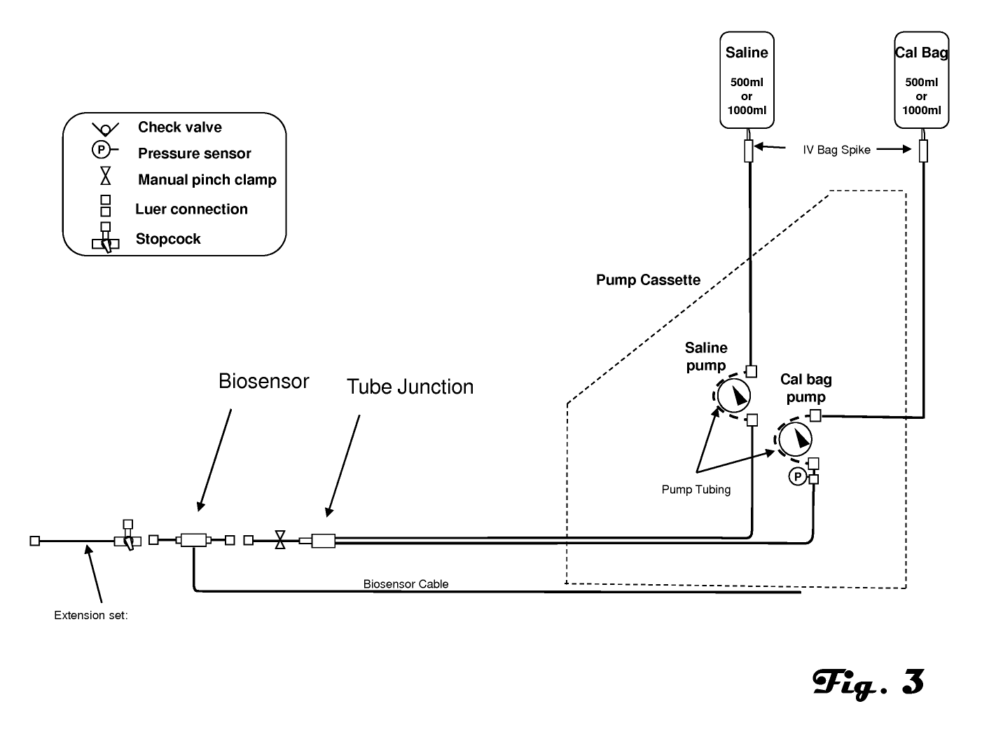

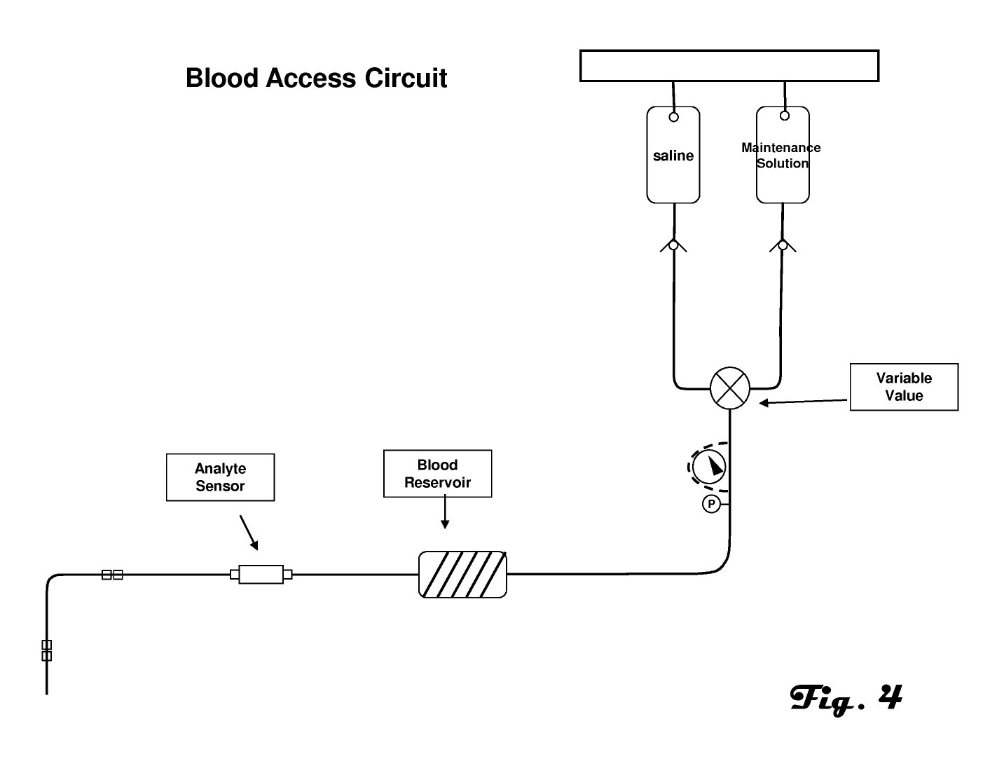

FIG. 3 is an illustration of an example embodiment where the sensor is located near the patient. The example automated blood analyte measurement system contains two fluid bags providing for at least two different calibration points, labeled as saline and cal bag. In use, the analyte sensor can be exposed to a zero or predetermined glucose concentration via fluid from the saline bag. A second glucose concentration can be provided via fluid from the calibration solution bag. The example system in FIG. 3 provides the opportunity for calibration of the device with a known maintenance fluid while concurrently minimizing the infusion of the maintenance fluid into the patient. In the example system, the calibration solution can be pumped through the circuit so that both tubes going to the sensor are filled with undiluted calibration solution. For example, the cal pump can be operated in a manner towards the patient and the saline pump can operate at a similar rate away from the patient. The fluid can go to a waste outlet (not shown) as needed. Alternately, the tubing can serve as sufficient reservoir for fluid that is undesirable to infuse into the patient, for example when the time of application of the apparatus is not overly long. When the tube junction contains an appropriate calibration solution, the pumps can be activated so as to push the calibration solution to the sensor. The sensor can be calibrated. To re-fill the circuit with a second calibration solution or a saline without glucose the saline pump can be operated in a manner towards the patient and the cal pump can operate at a similar rate away from the patient. This will result in a second solution near the tube junction. Again the solution can be moved to the sensor by operating both pumps toward the sensor or patient. The total amount of saline infused into the subject is dramatically reduced by the use of this “loop” circuit. Such a process can enable the effective calibration of the glucose sensor. Such a system also provides the opportunity to clean or maintain circuit performance with additives where minimizing the amount of infusion into the subject is desired.

The systems shown FIGS. 2 and 3 are also compatible with use of citrate as an anticoagulant. One example embodiment places citrate in the saline bag, since that is the fluid that makes the most contact with the blood. Contact with citrate effectively anticoagulates the blood during operation of the circuit. If there are concerns regarding binding of calcium at a high level, calcium can be added to the maintenance bag and infused into the patient during those periods between measurements.

FIG. 4 shows a different implementation of a two level sensor calibration system. The example system in FIG. 4 enables the analyte sensor to be exposed to at least two known glucose concentrations. The variable valve can be a simple stopcock where the solution provided to the analyte sensor is 100% maintenance solution or 100% saline solution. In other embodiments a variable valve can provide for controlled mixing of these two fluid solutions to create multiple glucose concentrations.

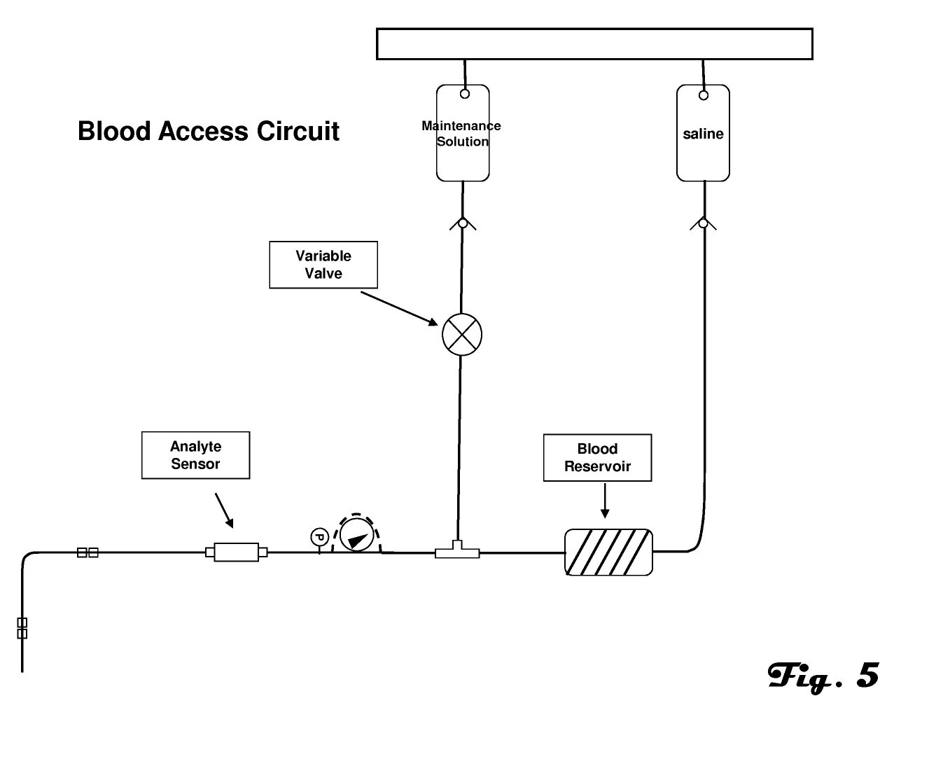

FIG. 5 is an illustration of an example embodiment which enables mixing of glucose into blood obtained from the patient. This example embodiment enables calibration of the analyte sensor at two known glucose concentrations, defined by the maintenance solution and the saline solution. In addition to providing the glucose sensor with non-blood based calibration solutions this system can also enable the calibration of the device using blood. In operation the blood sample can be withdrawn from the patient and exposed to the analyte sensor. Following this baseline measurement a predetermined amount of glucose can be added to the blood as it is pushed back towards the patient. This additive amount enables recalibration of the sensor with a blood based sample with a known additional amount of glucose. It is recognized that the system has the ability to create multiple glucose levels in both saline based calibration standards as well as defined different blood based calibration standards. The ability to manage the amount of mixing occurring at the T-junction and the corresponding glucose concentration at the analyte sensor can be controlled by the variable valve and pump. A blood reservoir is shown in the figure; in practice, such a reservoir can be any structure that allows blood be drawn past the point at which calibration fluid may be mixed with the blood, for example a length of tubing, a bag, fluid space within a pump, and a coil of tubing can all be suitable.

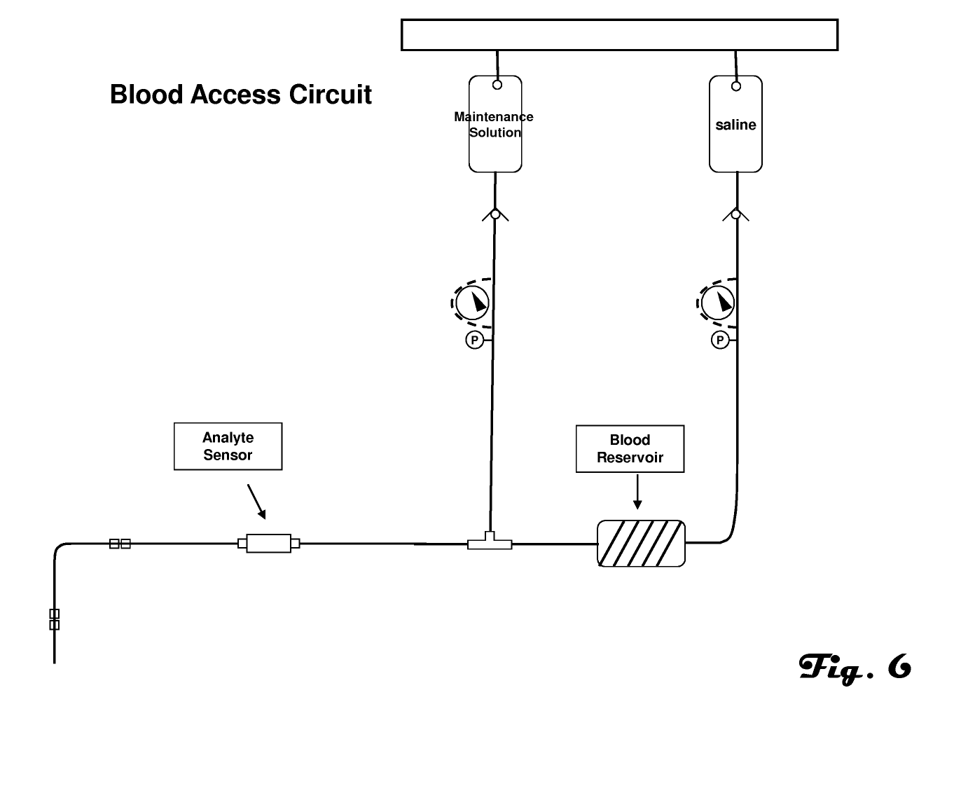

FIG. 6 is an illustration of an example embodiment with similar characteristics as those described in FIG. 5. The example embodiment in FIG. 6 contains two pumps. As shown in FIG. 6, these pumps are peristaltic pumps. Peristaltic pumps enable bidirectional flow as well as support stopped flow conditions. The example embodiment in FIG. 6 has the ability to perform a two point saline based calibration as well as defined glucose additions to the blood sample. The two pumps and reservoir provide the opportunity for assuring good mixing of the glucose throughout the sample. The example shows the use of peristaltic pumps but other pump mechanisms can be used, for example gradient flow, pressurized bags and other pump devices.

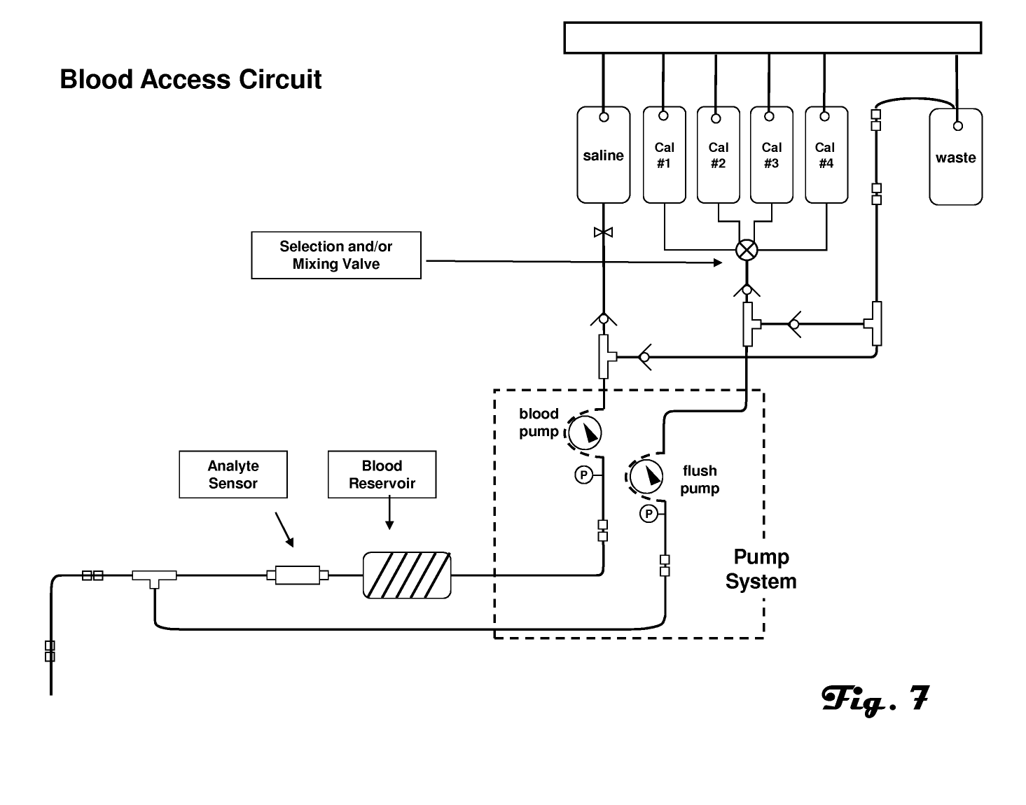

FIG. 7 is an illustration of an example embodiment of a blood access and measurement system suitable for use with the present invention. The example automated blood analyte measurement system contains a saline bag and a plurality of calibration bags. A selectable valve enables selection of the correct calibration solution or the mixing of several calibration solutions in a predetermined manner. In use, the analyte sensor can be exposed to a zero or predetermined glucose concentration via fluid from the saline bag and the calibration solutions. One or more additional glucose concentrations can be provided via fluid from the calibration solutions. The example system in FIG. 7 provides the opportunity for calibration of the device with one or more calibration solutions while concurrently minimizing the infusion of the calibration solutions into the patient. In the example system, the calibration solution can be pumped through the circuit and directly to waste without infusion into the patient. For example, the flush pump can be operated in a manner towards the patient and the blood pump can operate at a similar rate away from the patient. In this manner the analyte sensor is exposed to one or more calibration solutions but no fluid is infused into the patient. Following sensor calibration, fluid from the saline bag can be used to wash the circuit in a similar manner. Such a process can enable the effective calibration of a glucose or other analyte sensor. Such a system also provides the opportunity to clean or maintain circuit performance with additives where infusion into the subject is not desired. Following calibration, sensor performance can be validated by measuring an unused calibration solution or a unique mix of calibration solutions. The system also affords the ability to use one or more validation samples.

The system shown in FIGS. 7, 8, 9, and 10 are compatible with use of citrate as an anticoagulant. One example embodiment places citrate in the saline bag or in one of the calibration solutions, since that the fluid that makes the most contact with the blood. Contact with citrate effectively anticoagulates the blood during operation of the circuit. If there are concerns regarding binding of calcium at a high level, calcium can be added to the maintenance bag and infused into the patient during those periods between measurements.

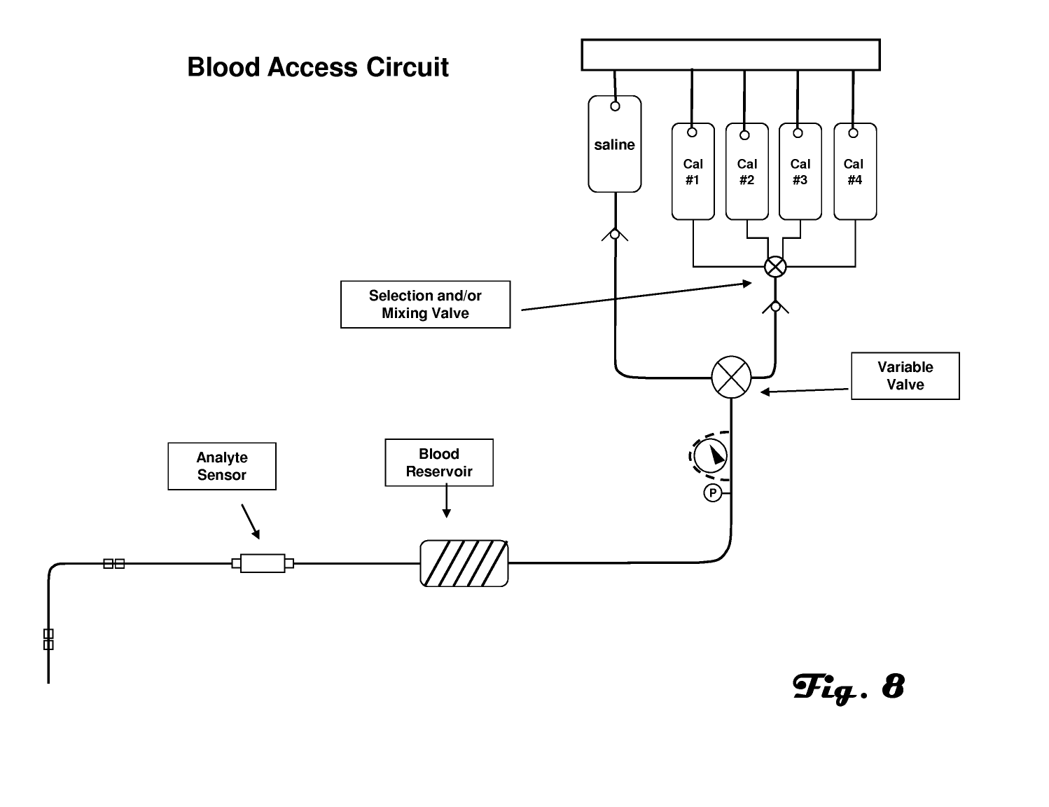

FIG. 8 is an illustration of an example implementation of a multi-level sensor calibration system. The example system in FIG. 8 enables the analyte sensor to be exposed to one or more calibration solutions. The variable valve can be a simple stopcock where the solution provided to the analyte sensory is 100% maintenance solution or 100% saline solution. A selection or mixing valve enables the selection of a particular calibration solution to be used or the creation of a determined mixture of calibration solutions. A variable valve can provide for controlled mixing of the fluid solutions to create multiple analyte concentrations.

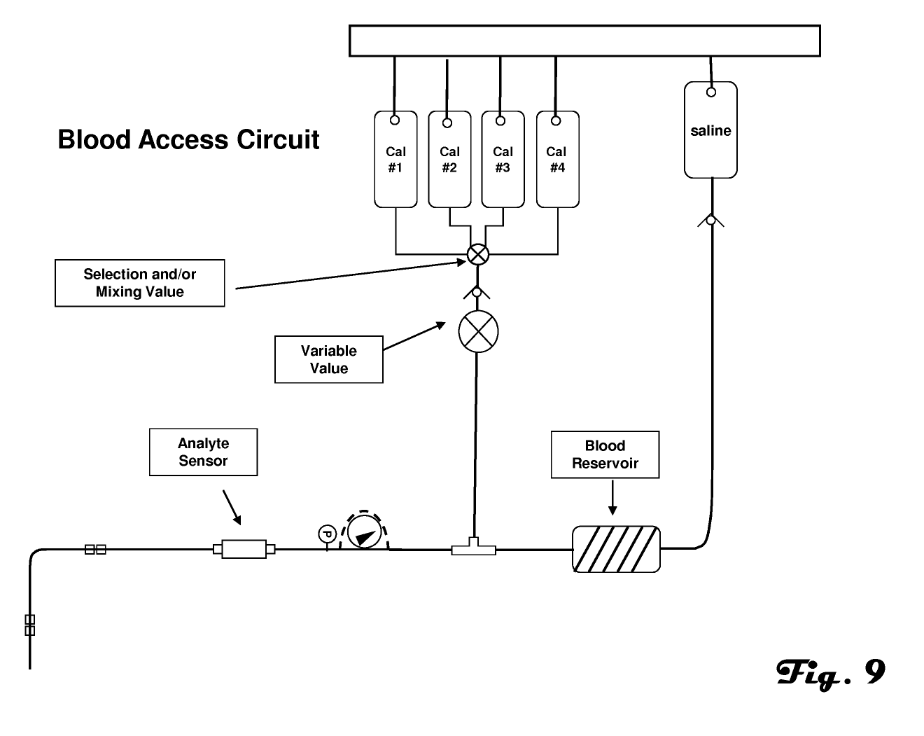

FIG. 9 is an illustration of an example embodiment which enables mixing of glucose into blood obtained from the patient. This example embodiment enables calibration of the analyte sensor at one or more known analyte concentrations, defined by the maintenance solution and the calibration solutions. The set of calibration solutions can allow calibration at a plurality of different analyte concentrations. In addition to providing the glucose sensor with non-blood based calibration solutions this system can also enable the calibration of the device using blood. In operation the blood sample can be withdrawn from the patient and exposed to the analyte sensor. Following this baseline measurement a predetermined amount of glucose can be added to the blood as it is pushed back towards the patient. The embodiment also provides the ability to add a plurality of calibration solutions to the blood sample. This ability to add calibration solutions to the blood sample enables recalibration of the sensor. It is recognized that the system has the ability to create multiple glucose levels in both saline based calibration standards as well as defined different blood based calibration standards. The ability to manage the amount of mixing occurring at the T-junction and the corresponding glucose concentration at the analyte sensor can be controlled by the variable valve and pump. The embodiment also provides the ability to create multiple validation levels both in saline-based solutions and in blood-based solutions.

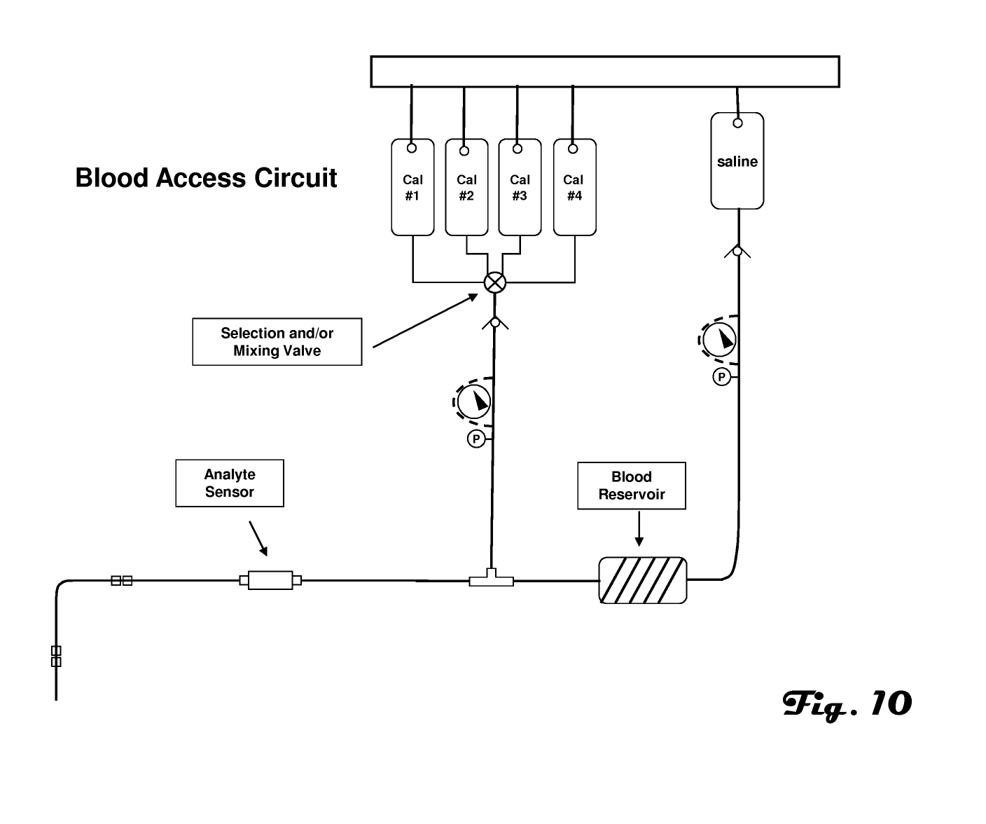

FIG. 10 is an illustration of an example embodiment with similar characteristics as those described in FIG. 9. The example embodiment in FIG. 10 contains two pumps and a selection and/or mixing valve associated with the calibration solutions. The selection and/or mixing valve can comprise a variety of embodiments, including a simple selection valve and a multipath system that enables mixing in a controlled manner. As shown in FIG. 10, these pumps are peristaltic pumps. Peristaltic pumps enable bidirectional flow as well as support stopped flow conditions. The example embodiment in FIG. 10 has the ability to perform a two point saline based calibration as well as defined glucose additions to the blood sample. The two pumps and reservoir provide the opportunity for assuring good mixing of the glucose throughout the sample.

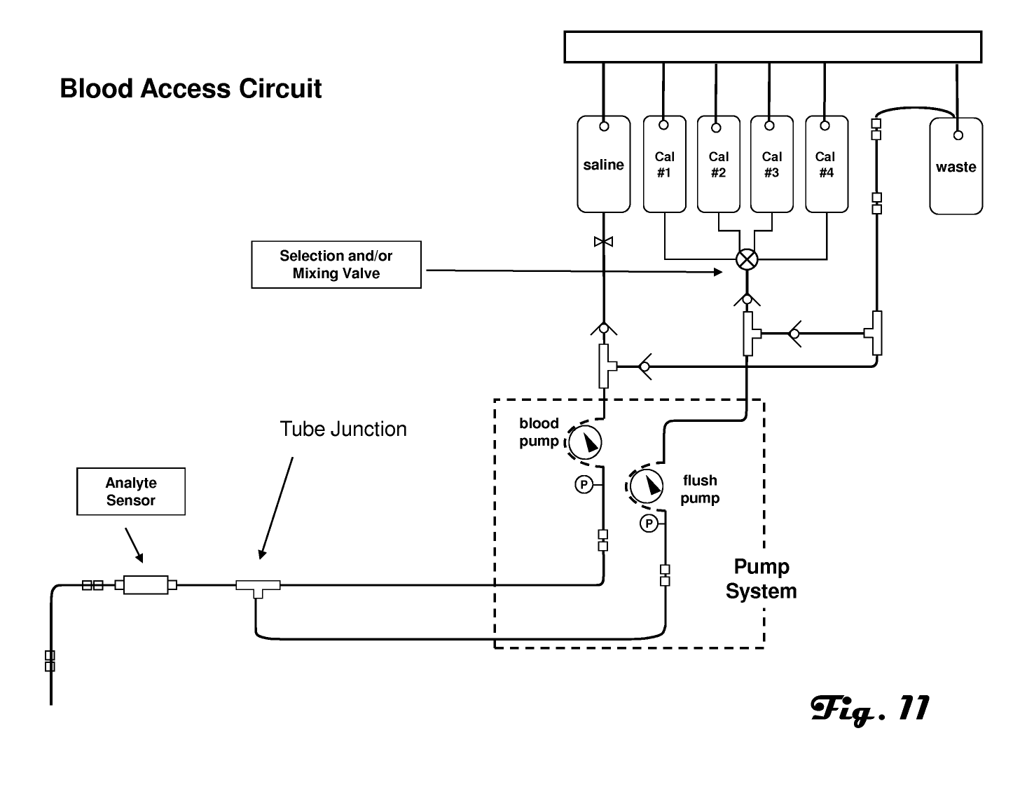

FIG. 11 is an illustration where the sensor is located near the patient and where the tube junction between the blood pump and saline pump is located distal the sensor. The example automated blood analyte measurement system contains a saline bag and a plurality of calibration bags. A selectable valve enables selection of the correct calibration solution or the mixing of several calibration solutions in a predetermined manner. In use, the analyte sensor can be exposed to a zero or predetermined glucose concentration via fluid from the saline bag and the calibration solutions. One or more additional glucose concentrations can be provided via fluid from the calibration solutions. The example system in FIG. 11 provides the opportunity for calibration of the device with one or more calibration solutions while concurrently minimizing the infusion of the calibration solutions into the patient. The overall fluid amount to the patient is minimized by moving the various saline or calibration fluids to the tube junction and only when the appropriate fluid is present near the tube junction is the solution moved to the sensor. For example, the calibration solution #1 can be pumped through the circuit so that the fluid at the tube junction is appropriate for calibration of the sensor. This can be accomplished by having the flush pump operate towards the patient and the blood pump operate at a similar rate away from the patient. The fluid can go to waste via a check value arrangement. When the tube junction contains an appropriate calibration solution, the pumps can be activated so as to push the calibration solution to the sensor, and the sensor calibrated. This fundamental process can be repeated for various calibration solutions and for saline. Thus, the patient only receives a small amount solution, approximately the volume between the tube-junction and the sensor. If no such loop system were employed the subject would receive larger volumes associated with the mixing or transition zone. The mixing or transition zone is the volume where two different solutions mix together. This occurs with or without movement but of a significant volume when solutions are pumped through tubing. Such a process enables the effective calibration of the glucose sensor. Such a system also provides the opportunity to clean or maintain circuit performance with additives where minimizing the amount of infusion into the subject is desired. Following calibration, sensor performance can be validated by measuring an unused calibration solution or a unique mix of calibration solutions. The system also affords the ability to use one or more validation samples. One of skill in the art can appreciate the fact that the number of calibration solutions can be varied from one to many with operation similar that defined above.

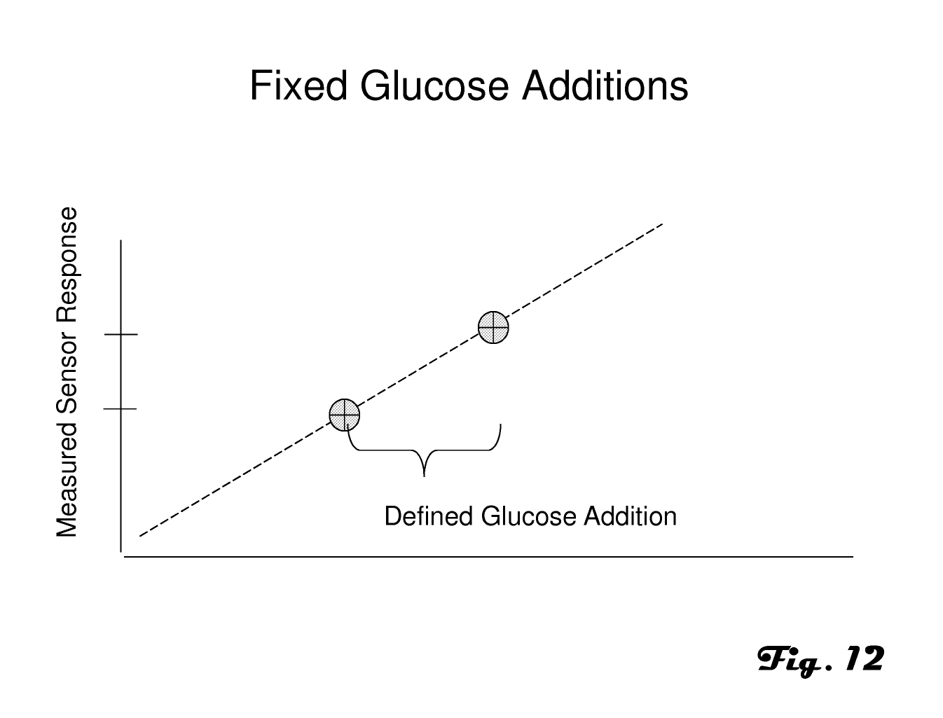

FIG. 12 shows a simplistic example of how a fixed glucose addition to a sample of unknown glucose concentration enables calibration of the device. This concept can be extrapolated to multiple additions or even a response surface mapping with continuous increase or decrease in glucose concentration.

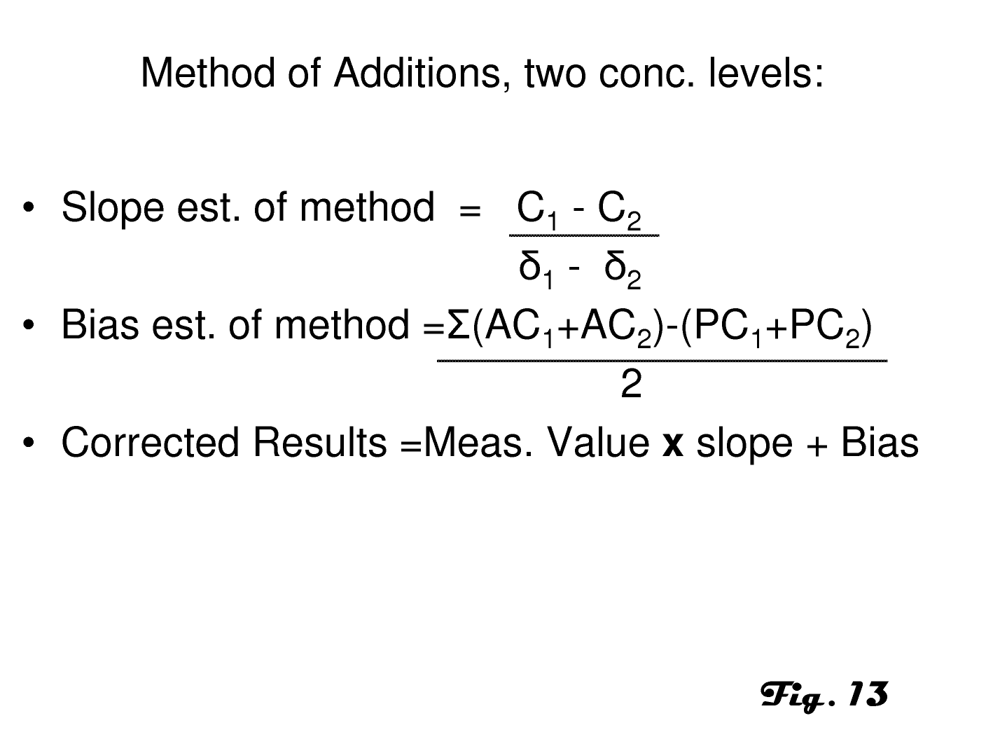

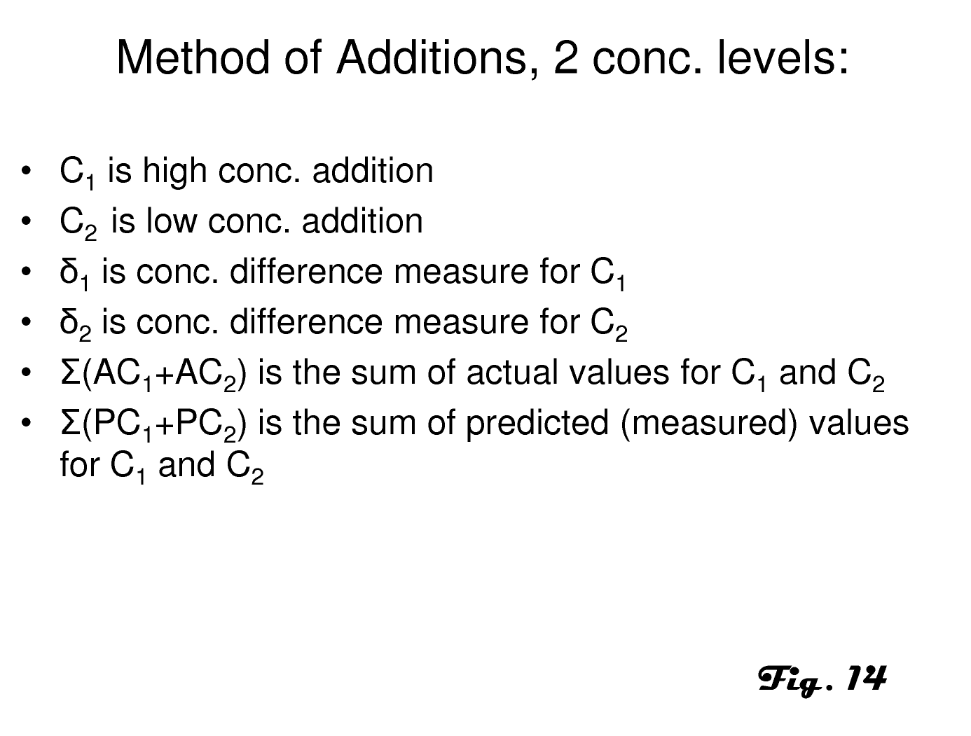

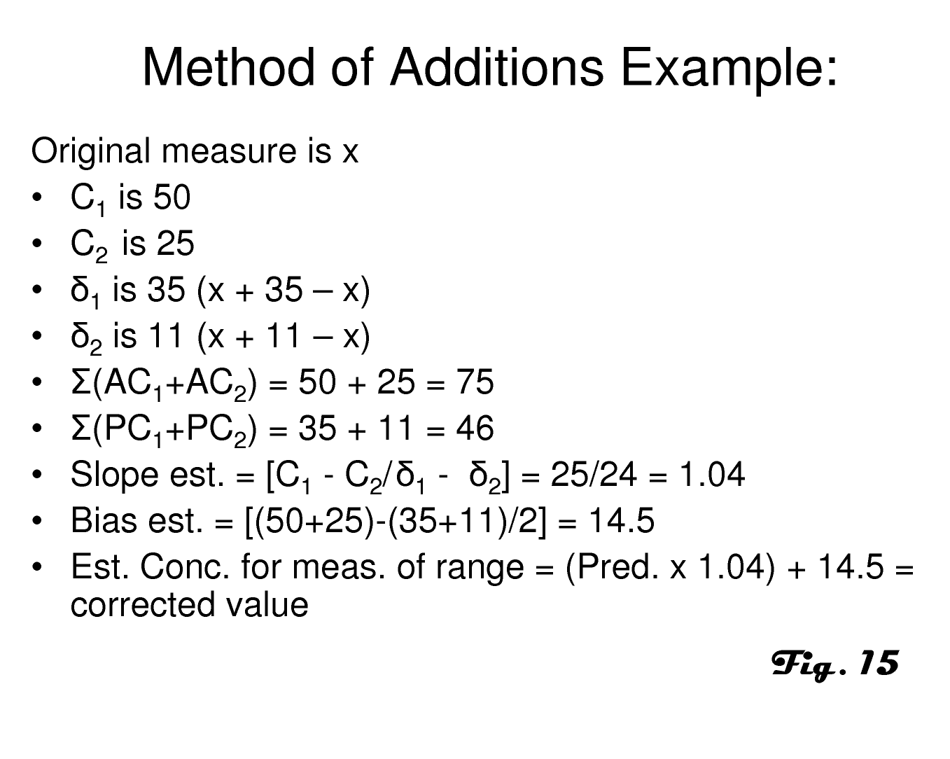

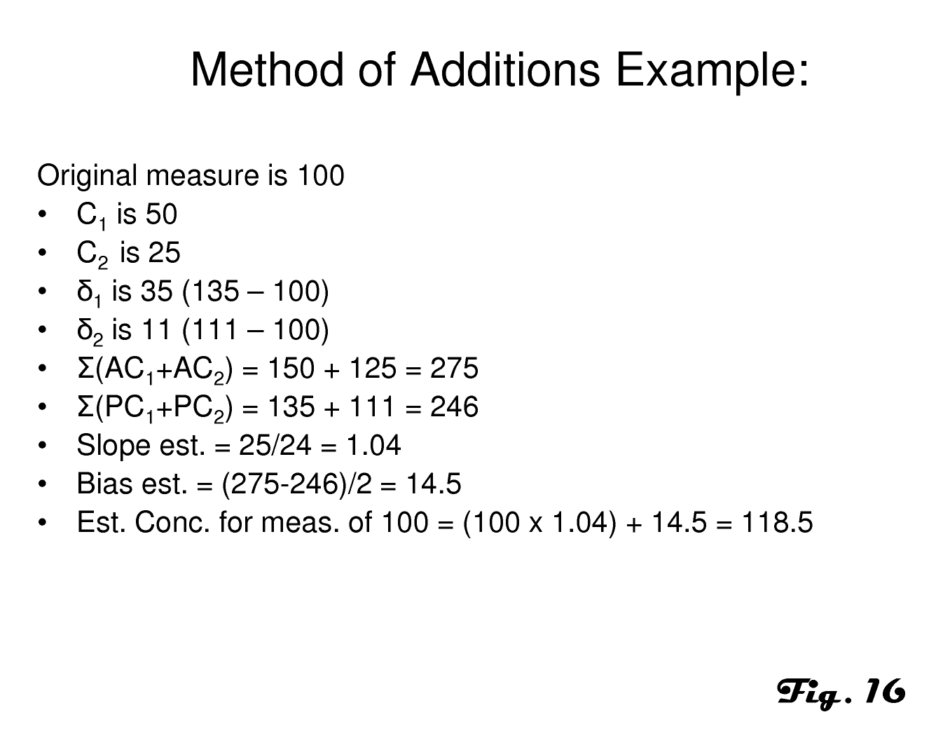

FIGS. 13, 14, 15 and 16 show several examples of how the methods of additions can be used in calibration of the sensor. In FIG. 15, the method is applied where the concentration of the sample is not known but the amount of change to the sample is defined. This process can be used with the current invention to provide for accurate calibration.

In a first example method, the invention provides a method of calibrating an automated analyte measurement system that removes blood from a patient for measurement, comprising passing calibration fluid having at least two different analyte concentrations by an analyte sensor while infusing substantially none of at least one of such calibration fluids into the patient. In such an example, that sensor and calibration fluid can be maintained in a sterile condition.

In a second example method, the present invention provides a method of validating the performance of an automated analyte measurement system, comprising calibrating the system according to the method of claim 1, then determining the sensor response to a calibration fluid having an analyte concentration different from those used in calibration while infusing substantially none of such calibration fluid into the patient.

In a first example apparatus, the present invention provides an apparatus for the measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element; a first fluid source in fluid communication with the second port of the analyte sensor; a second fluid source in fluid communication with the first port of the analyte sensor; a first pump mounted with the apparatus so as to move fluid from the first fluid source towards or away from the analyte sensor; a second pump mounted with the apparatus so as to urge fluid from the second fluid source toward or away from the analyte sensor; and a waste outlet in fluid communication with at least one of the first and second ports of the analyte sensor; wherein at least one of the first fluid source and the second fluid source contains a fluid having a first known analyte concentration suitable for calibration of the analyte sensor.

In an apparatus like the first example apparatus, the first fluid source can contain a fluid having a first known analyte concentration suitable for calibration of the analyte sensor, and wherein the second fluid source contains a fluid having a second known analyte concentration, different from the first known analyte concentration, suitable for calibration of the analyte sensor.

In an apparatus like the first example apparatus, the apparatus can further comprise a third fluid source in fluid communication with at least one of the first port or the second port of the analyte sensor, and containing a fluid having a second known analyte concentration, different from the first known analyte concentration, suitable for calibration of the analyte sensor.

In an apparatus like the first example apparatus, the apparatus can further comprise a selection or mixing valve mounted between either the first fluid source or the second fluid source and the corresponding port of the analyte sensor, and further comprising a third fluid source in fluid communication with the variable mixing valve, and containing a fluid having a second known analyte concentration, different from the first known analyte concentration, suitable for calibration of the analyte sensor.

In a second example apparatus, the present invention provides an apparatus for measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element and separated therefrom by a fluid passage having a first length; a tubing junction comprising first, second, and third ports, the first port in fluid communication with the second port of the analyte sensor and separated therefrom by a fluid passage having a second length; a first fluid source in fluid communication with the second port of the tubing junction and separated therefrom by a fluid passage having a third length, where the sum of the second and third lengths is greater than the first length; a second fluid source in fluid communication with the third port of the tubing junction; a first pump mounted with the apparatus so as to urge fluid from the first fluid source towards or away from the tubing junction; and a second pump mounted with the apparatus so as to urge fluid from the second fluid source toward or away from the tubing junction; wherein the first fluid source contains a fluid having a first known analyte concentration suitable for calibration of the analyte sensor.

In an apparatus like the second example apparatus, the second fluid source can contain a fluid having a second known analyte concentration suitable for calibration of the analyte sensor.

In an apparatus like the second example apparatus, the apparatus can further comprise a selection or mixing valve mounted between the first fluid source and the tubing junction, and further comprising a third fluid source having a fluid having a third known analyte concentration suitable for calibration of the analyte sensor mounted in fluid communication with the selection or mixing valve.

In a third example apparatus, the present invention provides an apparatus for the measurement of one or more analytes in blood withdrawn from a patient, comprising: a patient connection fluid passage element configured to be placed in fluid communication with the vascular system of a patient; an analyte sensor having first and second ports, the first port in fluid communication with the patient connection fluid passage element; a reservoir in fluid communication with the second port of the analyte sensor; a first fluid source in fluid communication with the second port of the analyte sensor, wherein the first fluid source contains a fluid having a first known analyte concentration suitable for calibration of the analyte sensor; a first pump mounted with the apparatus so as to urge fluid from the first fluid source towards or away from the analyte sensor; and a second pump mounted with the apparatus so as to urge fluid from the reservoir toward or away from the analyte sensor.

In an apparatus like the third example apparatus, the apparatus can further comprise a second fluid source in fluid communication with the second port of the analyte sensor, wherein the second fluid source contains a fluid having a second known analyte concentration, different from the first known analyte concentration, suitable for calibration of the analyte sensor.

In third example method, the present invention provides a method of calibrating an apparatus such as the first example apparatus, comprising operating the first and second pumps to flow fluid from the fluid source having a known analyte concentration past the sensor and to the waste outlet, and calibrating the analyte sensor responsive to its response to the fluid having the first known analyte concentration.

In a method like the third example method, wherein the apparatus further comprises a third fluid source in fluid communication with at least one of the first port or the second port of the analyte sensor, and containing a fluid having a second known analyte concentration, different from the first known analyte concentration, suitable for calibration of the analyte sensor, the method can further comprise operating the first and second pumps to flow fluid from the fluid source having a known analyte concentration past the analyte sensor and to the waste outlet, and operating the first and second pumps to flow fluid from the third fluid source past the analyte sensor and to the waste outlet, and calibrating the analyte sensor responsive to its response to the fluid having the first known analyte concentration and its response to the fluid having the second known analyte concentration.

In a method like the third example method, wherein the apparatus further comprises a selection or mixing valve mounted between either the first fluid source or the second fluid source and the corresponding port of the analyte sensor, and further comprising a third fluid source in fluid communication with the selection or mixing valve, and containing a fluid having a second known analyte concentration, different from the first known analyte concentration, suitable for calibration of the analyte sensor, the method can further comprise configuring the selection or mixing valve to pass either of its input fluids or a combination of its input fluids to deliver a first calibration fluid having a first calibration analyte concentration, and operating the first and second pumps to flow the first calibration fluid past the analyte sensor and to the waste outlet, and configuring the selection or mixing valve to pass either of its input fluids or a combination of its input fluids to deliver a second calibration fluid having a second calibration analyte concentration different from the first calibration analyte concentration, and operating the first and second pumps to flow the second calibration fluid past the analyte sensor and to the waste outlet, and calibrating the analyte sensor responsive to its response to the first calibration fluid and its response to the second calibration fluid.

In a method like the third example method, the method can be practiced such that substantially none of the fluid is infused into the patient. In a method like the third example method, the method can be practiced such that an amount of fluid less than the amount that would be likely to cause harm to the patient can be infused into the patient.

In fourth example method, the present invention provides a method of calibrating an apparatus such as in the second example apparatus, comprising operating the first and second pumps to flow fluid from the first fluid source past the analyte sensor while infusing into the patient a volume less than the volume defined by the fluid passage between the tubing junction and the first fluid source, and calibrating the analyte sensor responsive to its response to the fluid.

In a method like the fourth example method, wherein the second fluid source contains a fluid having a second known analyte concentration, different from the first known analyte concentration, suitable for calibration of the analyte sensor, the method can further comprise operating the first and second pumps to flow fluid from the second fluid source past the analyte sensor while infusing into the patient a volume less than the volume defined by the fluid passage between the tubing junction and the second fluid source, and calibrating the analyte sensor responsive to its response to the fluid from the first fluid source and its response to fluid from the second fluid source.

In a method like the fourth example method, wherein the apparatus further comprises a selection or mixing valve mounted between the first fluid source and the tubing junction, and further comprising a third fluid source having a fluid having a third known analyte concentration suitable for calibration of the analyte sensor mounted in fluid communication with the selection or mixing valve, the method can further comprise configuring the selection or mixing valve to deliver a first calibration fluid comprising fluid from the first fluid source, fluid from the third fluid source, or a combination thereof, and operating the pumps to flow the first calibration fluid past the analyte sensor while infusing into the patient a volume less than the volume defined by the fluid passage between the tubing junction and the selection or mixing valve, and configuring the selection or mixing valve to deliver a second calibration fluid comprising fluid from the first fluid source, fluid from the third fluid source, or a combination thereof, and operating the pumps to flow the second calibration fluid past the analyte sensor while infusing into the patient a volume less than the volume defined by the fluid passage between the tubing junction and the selection or mixing valve, and calibrating the analyte sensor responsive to its response to the first and second calibration fluids.

In a fifth example method, the present invention provides a method of calibrating an apparatus such as the third example apparatus, comprising operating the first and second pumps to withdraw blood from the patient past the analyte sensor and into the reservoir, and operating the first and second pumps to draw blood from the reservoir and fluid from the first fluid source to present a mixture of blood from the reservoir and fluid from the first fluid source to the analyte sensor, and calibrating the analyte sensor responsive to its response to the blood and to the mixture of blood and the fluid from the first fluid source.

In a method like the fifth example method, wherein the apparatus further comprises a second fluid source in fluid communication with the second port of the analyte sensor, wherein the second fluid source contains a fluid having a second known analyte concentration, different from the first known analyte concentration, suitable for calibration of the analyte sensor, the method can further comprise operating the first and second pumps to draw blood from the reservoir and fluid from the second fluid source to present a mixture of blood from the reservoir and fluid from the second fluid source to the analyte sensor, and calibrating the analyte sensor responsive to its response to the blood and to the mixture of blood and the fluid from the first fluid source and to the mixture of blood and fluid from the second fluid source.

The present invention has been described as set forth herein. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A method of calibrating an automated analyte measurement system that removes blood from a patient for measurement, comprising passing calibration fluid having at least two different analyte concentrations by an analyte sensor while infusing substantially none of at least one of such calibration fluids into the patient.

2. A method as in claim 1, wherein the sensor and the calibration fluid are maintained in a sterile condition.

3. A method of validating the performance of an automated analyte measurement system, comprising calibrating the system according to the method of claim 1, then determining the sensor response to a calibration fluid having an analyte concentration different from those used in calibration while infusing substantially none of such calibration fluid into the patient.

4. An apparatus for the measurement of one or more analytes in blood withdrawn from a patient, comprising:
a. A patient connection fluid passage element configured to be placed in fluid communication with the vascular system of a patient;
b. An analyte sensor having first and second ports, the first port in fluid communication with the patient connection fluid passage element;
c. A first fluid source in fluid communication with the second port of the analyte sensor;
d. A second fluid source in fluid communication with the first port of the analyte sensor;
e. A first pump mounted with the apparatus so as to move fluid from the first fluid source towards or away from the analyte sensor;
f. A second pump mounted with the apparatus so as to urge fluid from the second fluid source toward or away from the analyte sensor; and
g. A waste outlet in fluid communication with at least one of the first and second ports of the analyte sensor;
h. Wherein at least one of the first fluid source and the second fluid source contains a fluid having a first known analyte concentration suitable for calibration of the analyte sensor.

5. An apparatus as in claim 4, wherein the first fluid source contains a fluid having a first known analyte concentration suitable for calibration of the analyte sensor, and wherein the second fluid source contains a fluid having a second known analyte concentration, different from the first known analyte concentration, suitable for calibration of the analyte sensor.

6. An apparatus as in claim 4, further comprising a third fluid source in fluid communication with at least one of the first port or the second port of the analyte sensor, and containing a fluid having a second known analyte concentration, different from the first known analyte concentration, suitable for calibration of the analyte sensor.

7. An apparatus as in claim 4, further comprising a selection or mixing valve mounted between either the first fluid source or the second fluid source and the corresponding port of the analyte sensor, and further comprising a third fluid source in fluid communication with the variable mixing valve, and containing a fluid having a second known analyte concentration, different from the first known analyte concentration, suitable for calibration of the analyte sensor.

8. A method of calibrating an apparatus as in claim 4, comprising operating the first and second pumps to flow fluid from the fluid source having a known analyte concentration past the sensor and to the waste outlet, and calibrating the analyte sensor responsive to its response to the fluid having the first known analyte concentration.

9. A method as in claim 8, wherein the apparatus further comprises a third fluid source in fluid communication with at least one of the first port or the second port of the analyte sensor, and containing a fluid having a second known analyte concentration, different from the first known analyte concentration, suitable for calibration of the analyte sensor, the method further comprising operating the first and second pumps to flow fluid from the fluid source having a known analyte concentration past the analyte sensor and to the waste outlet, and operating the first and second pumps to flow fluid from the third fluid source past the analyte sensor and to the waste outlet, and calibrating the analyte sensor responsive to its response to the fluid having the first known analyte concentration and its response to the fluid having the second known analyte concentration.

10. A method as in claim 8, wherein the apparatus further comprises a selection or mixing valve mounted between either the first fluid source or the second fluid source and the corresponding port of the analyte sensor, and further comprising a third fluid source in fluid communication with the selection or mixing valve, and containing a fluid having a second known analyte concentration, different from the first known analyte concentration, suitable for calibration of the analyte sensor, the method further comprising configuring the selection or mixing valve to pass either of its input fluids or a combination of its input fluids to deliver a first calibration fluid having a first calibration analyte concentration, and operating the first and second pumps to flow the first calibration fluid past the analyte sensor and to the waste outlet, and configuring the selection or mixing valve to pass either of its input fluids or a combination of its input fluids to deliver a second calibration fluid having a second calibration analyte concentration different from the first calibration analyte concentration, and operating the first and second pumps to flow the second calibration fluid past the analyte sensor and to the waste outlet, and calibrating the analyte sensor responsive to its response to the first calibration fluid and its response to the second calibration fluid.

11. An apparatus for measurement of one or more analytes in blood withdrawn from a patient, comprising:
a. A patient connection fluid passage element configured to be placed in fluid communication with the vascular system of a patient;
b. An analyte sensor having first and second ports, the first port in fluid communication with the patient connection fluid passage element and separated therefrom by a fluid passage having a first length;
c. A tubing junction comprising first, second, and third ports, the first port in fluid communication with the second port of the analyte sensor and separated therefrom by a fluid passage having a second length;
d. A first fluid source in fluid communication with the second port of the tubing junction and separated therefrom by a fluid passage having a third length, where the sum of the second and third lengths is greater than the first length;
e. A second fluid source in fluid communication with the third port of the tubing junction;
f. A first pump mounted with the apparatus so as to urge fluid from the first fluid source towards or away from the tubing junction; and
g. A second pump mounted with the apparatus so as to urge fluid from the second fluid source toward or away from the tubing junction;
h. Wherein the first fluid source contains a fluid having a first known analyte concentration suitable for calibration of the analyte sensor.

12. An apparatus as in claim 11, wherein the second fluid source contains a fluid having a second known analyte concentration suitable for calibration of the analyte sensor.

13. An apparatus as in claim 11, further comprising a selection or mixing valve mounted between the first fluid source and the tubing junction, and further comprising a third fluid source having a fluid having a third known analyte concentration suitable for calibration of the analyte sensor mounted in fluid communication with the selection or mixing valve.

14. A method of calibrating an apparatus as in claim 11, comprising operating the first and second pumps to flow fluid from the first fluid source past the analyte sensor while infusing into the patient a volume less than the volume defined by the fluid passage between the tubing junction and the first fluid source, and calibrating the analyte sensor responsive to its response to the fluid.

15. A method as in claim 14, wherein the second fluid source contains a fluid having a second known analyte concentration, different from the first known analyte concentration, suitable for calibration of the analyte sensor, the method further comprising operating the first and second pumps to flow fluid from the second fluid source past the analyte sensor while infusing into the patient a volume less than the volume defined by the fluid passage between the tubing junction and the second fluid source, and calibrating the analyte sensor responsive to its response to the fluid from the first fluid source and its response to fluid from the second fluid source.

16. A method as in claim 14, wherein the apparatus further comprises a selection or mixing valve mounted between the first fluid source and the tubing junction, and further comprising a third fluid source having a fluid having a third known analyte concentration suitable for calibration of the analyte sensor mounted in fluid communication with the selection or mixing valve, the method further comprising configuring the selection or mixing valve to deliver a first calibration fluid comprising fluid from the first fluid source, fluid from the third fluid source, or a combination thereof, and operating the pumps to flow the first calibration fluid past the analyte sensor while infusing into the patient a volume less than the volume defined by the fluid passage between the tubing junction and the selection or mixing valve, and configuring the selection or mixing valve to deliver a second calibration fluid comprising fluid from the first fluid source, fluid from the third fluid source, or a combination thereof, and operating the pumps to flow the second calibration fluid past the analyte sensor while infusing into the patient a volume less than the volume defined by the fluid passage between the tubing junction and the selection or mixing valve, and calibrating the analyte sensor responsive to its response to the first and second calibration fluids.

17. An apparatus for the measurement of one or more analytes in blood withdrawn from a patient, comprising:
a. A patient connection fluid passage element configured to be placed in fluid communication with the vascular system of a patient;
b. An analyte sensor having first and second ports, the first port in fluid communication with the patient connection fluid passage element;
c. A reservoir in fluid communication with the second port of the analyte sensor;
d. A first fluid source in fluid communication with the second port of the analyte sensor, wherein the first fluid source contains a fluid having a first known analyte concentration suitable for calibration of the analyte sensor;
e. A first pump mounted with the apparatus so as to urge fluid from the first fluid source towards or away from the analyte sensor; and
f. A second pump mounted with the apparatus so as to urge fluid from the reservoir toward or away from the analyte sensor.

18. An apparatus as in claim 17, further comprising a second fluid source in fluid communication with the second port of the analyte sensor, wherein the second fluid source contains a fluid having a second known analyte concentration, different from the first known analyte concentration, suitable for calibration of the analyte sensor.

19. A method of calibrating an apparatus as in claim 17, comprising operating the first and second pumps to withdraw blood from the patient past the analyte sensor and into the reservoir, and operating the first and second pumps to draw blood from the reservoir and fluid from the first fluid source to present a mixture of blood from the reservoir and fluid from the first fluid source to the analyte sensor, and calibrating the analyte sensor responsive to its response to the blood and to the mixture of blood and the fluid from the first fluid source.

20. A method as in claim 19, wherein the apparatus further comprises a second fluid source in fluid communication with the second port of the analyte sensor, wherein the second fluid source contains a fluid having a second known analyte concentration, different from the first known analyte concentration, suitable for calibration of the analyte sensor, the method further comprising operating the first and second pumps to draw blood from the reservoir and fluid from the second fluid source to present a mixture of blood from the reservoir and fluid from the second fluid source to the analyte sensor, and calibrating the analyte sensor responsive to its response to the blood and to the mixture of blood and the fluid from the first fluid source and to the mixture of blood and fluid from the second fluid source.

21. A method as in claim 8, wherein substantially none of the fluid is infused into the patient.
